(19) United States
(12) Patent Application Publication
(10) Pub. No.: US 2016/0073932 A1
(43) Pub. Date: Mar. 17, 2016

(54) Device For Measuring The Diameter Of An Aortic Valve

(71) Applicants: Bernard PAIN, Monistrol sur Loire (FR); Marco VOLA, Saint Priest en Jarez (FR)

(72) Inventors: Bernard Pain, Monistrol sur Loire (FR); Marco Vola, Saint Priest en Jarez (FR)

(21) Appl. No.: 14/786,897

(22) PCT Filed: Apr. 16, 2014
(86) PCT No.: PCT/FR2014/050922

§ 371 Date: Oct. 23, 2015

(30) Foreign Application Priority Data
Apr. 25, 2013 (FR) 1353762

(51) Int. Cl. A61B 5/107; A61B 5/00; A61B 1/313; A61B 5/02

(52) U.S. Cl. CPC A61B 5/1076; A61B 5/02007; A61B 5/6876; A61B 1/3137; A61B 5/742

(57) ABSTRACT

The device includes a body having the general shape of a cylinder so as to be engaged in a trocar that is introduced into the aortic duct, the body having, at one of its ends that is accessible from outside the trocar, a maneuvering member secured to a measuring means which is present at the other end of the body and which is able to be deployed diametrically under the effect of an action exerted on the maneuvering member in order to come into contact with the wall of the aortic valve.

### FIELD OF THE INVENTION

The invention concerns the technical sector of measuring devices, applicable in the medical field to measure the diameter of an aortic valve, advantageously in the field of endoscopic surgery.

### BACKGROUND OF THE INVENTION

Note that the aortic valve is situated at the outlet of the left ventricle at the origin of the aorta and opens on each beat of the heart. For a variety of medical reasons, it is sometimes necessary to replace the aortic valve which can, for instance, become gradually more rigid, calcified, and shrunken . . . . It is replaced by an artificial valve.

Obviously, the aortic valve diameter differs in each individual. In other words, the latter can be replaced by an artificial valve having a diameter corresponding to the valve to be replaced. This makes it particularly important to measure accurately the diameter of the orifice to select the right size of valve adapted to the aortic ring.

The current devices used for measuring the diameter of the valve are rigid and cannot therefore be reduced. Generally, the surgeon makes an incision and after removing the aortic valve to be replaced, makes a radiographic measurement. However, there are mechanical components, such as the elasticity of the aortic ring, which are entirely unforeseeable, making mechanical calibration essential, by a surgical gesture with annular decalcification.

Accordingly, it is evident that the measuring technique now used will not allow mini-invasive surgery to be performed while considering, for instance, that the diameters of the trocars used today for this type of operation are around 18 to 20 mm. But it is apparent that the diameter of the aortic valve can vary between 20 and 40 mm. This makes it impossible to pass a 40 mm measuring gauge through a trocar which is, in fact, 20 mm at the most.

One solution appears in the information given in document US 2010/0249661 describing a device measuring the diameter of a valve under the effect of the deployment of a shape-memory ribbon whose resistance makes it possible to check that the surgeon is in contact with the walls. In other words, it is not the surgeon who controls this resistance.

### SUMMARY OF THE INVENTION

The purpose of the invention is to remedy these drawbacks in a reliable, simple, efficient and rational way.

The issue that the invention proposes to resolve is to be able to measure the diameter of the aortic valve by passing through the smallest possible hole, ideally using, for instance, a trocar, of a type with which the skilled person is totally familiar, so that the surgeon is able to control the measurement when he feels that a certain resistance is encountered, corresponding to the diameter of the valve.

In order to resolve an issue like this, an aortic valve diameter measuring device has been designed and developed, comprising a body of a generally cylindrical shape designed to be engaged into a trocar inserted into the aortic conduit, said body having at one of its ends accessible from the outside of the trocar, a maneuvering device connected at the other end of the body, to a measuring means consisting of a flexible ribbon mounted in conjunction with a rotating shaft operated by the maneuvering device and a fixed part of the body arranged to be concentric with the said shaft and from which the said ribbon is deployed under the effect of action on the said maneuvering device to increase the diameter of the ribbon until it comes into contact with the walls of the aortic valve until such a time as rotation becomes impossible, generating resistance corresponding to the diameter of the valve.

By virtue of these characteristics, it means that with the measuring means in the non-deployed condition, the body can be engaged in the trocar and, once it is inside the aortic conduit, it is simply necessary to adjust the maneuvering device to cause the combined diametrical development of said measuring means until it abuts on the internal diameter of the aortic ring whose maximum diameter can be in the region of 40 mm.

It is thus possible to increase gradually from 20 to 40 mm.

In an important manner, it is the surgeon who determines that the resistance is reached, corresponding to the desired measurement.

In one embodiment, one end of the ribbon is attached in a slot which has a rotating shaft on which it is wound and extends through an opening in the fixed part of the body so as to be attached at the other end to the outside of the said part by being wound on it, corresponding to the non-deployed position of the ribbon.

In this non-deployed position of the ribbon, the latter is spiral-wound about the corresponding part of the rotating shaft inside the fixed part of the body.

According to another characteristic, the fixed part of the body comprises a circular bearing surface which serves as a stator, such bearing surface having transversal shoulders for guiding the ribbon at either end.

To resolve the issue of allowing the shaft to be driven in rotation for the deployment of the ribbon in one direction and its rewinding in the other direction, the maneuvering device has a rotating knob mounted to the end of the body at the opposite end of the ribbon winding fixed part. The winding shaft is assembled so as to be free to rotate inside the body and is locked in its translation in the said body.

To resolve the problem posed of enabling the surgeon to know instantly the measured diameter of the aortic valve, the device has visual indication means of the measured diameter corresponding to the deployment of the ribbon.

In one embodiment, the visual indication means are arranged on the ribbon in order to be read by an endoscope.

In another embodiment, the visual indication means comprise a graduation made on the body of the maneuvering device. The graduation is aligned with a window in the maneuvering device.

### BRIEF DESCRIPTION OF THE DRAWINGS

The invention is illustrated in greater detail in the figures of the attached drawings in which:

FIG. 1 is a perspective view, in the non-deployed position, of the measuring ribbon corresponding to the insertion of the device through a trocar.

FIG. 2 is a perspective view of one end of the body considered at the fixed part from which the ribbon is deployed, which is shown in the non-deployed position corresponding to the position illustrated in FIG. 1.

FIG. 3 is a partial perspective view of the end of the body of the considered device, at the maneuvering knob.

FIG. 4 is a perspective view corresponding to FIG. 1 with the measuring ribbon in the deployed position.

FIG. 5 is a perspective view corresponding to FIG. 2 showing the ribbon in the deployed position for measuring the diameter of the aortic valve.

### DETAILED DESCRIPTION OF THE INVENTION

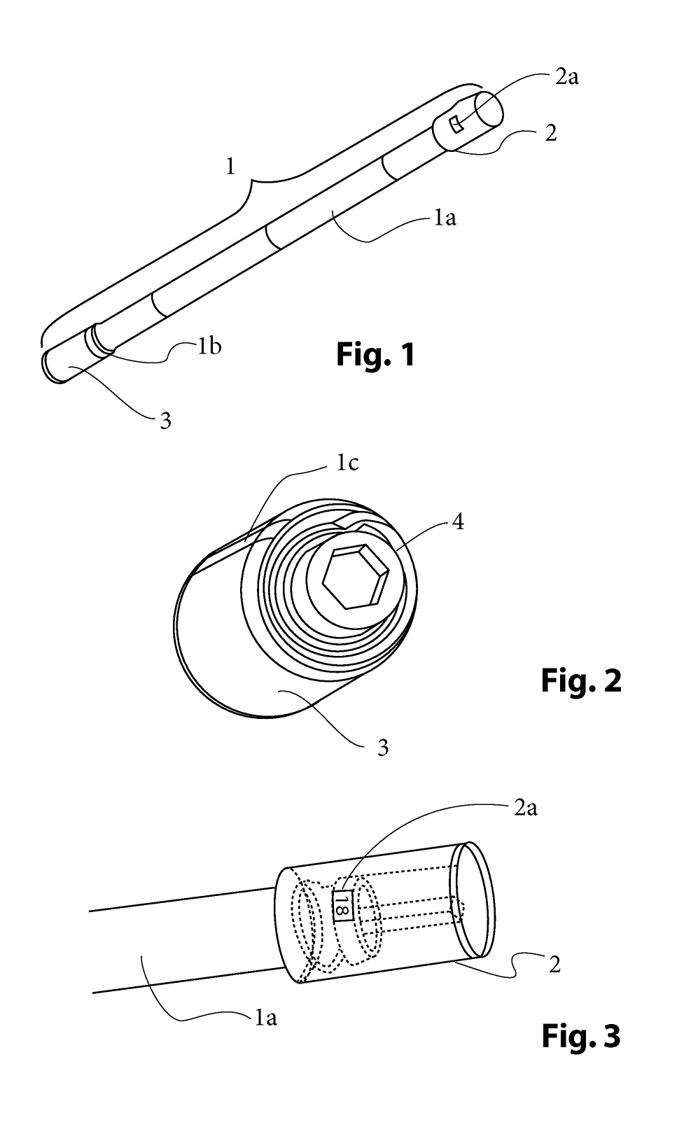

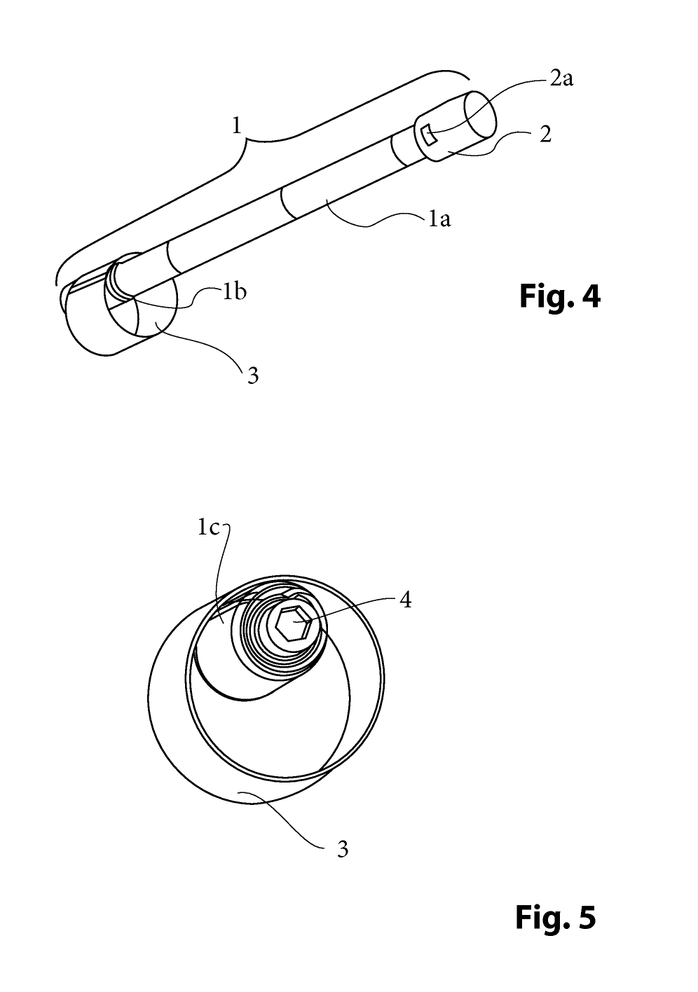

As shown more particularly in FIGS. 1 and 4, the device includes a body (1), generally of a cylindrical shape, to be engaged through a trocar intended to be inserted into the aortic conduit. For instance, body (1) has a diameter of around 18 mm corresponding to the diameter of the trocars used today. Body (1), at one of its accessible ends from the outside of the trocar, has a maneuvering device (2).

As will be indicated in the continuation of the description, this device (2) consists of a rotating knob. At the opposite end of the maneuvering device (2), body (1) has a measuring means (3) suitable for diametrical deployment under the effect of action applied to maneuvering device (2).

Measuring means (3) is a flexible ribbon mounted in conjunction with a rotating shaft (4) operated by maneuvering device (2) and, also, on the one hand a fixed part of body (1c) arranged to be concentric to said shaft (4). Ribbon (3) is deployed linearly from fixed part (1c) of the body.

As shown in FIGS. 2 and 5, one end of ribbon (3) is attached for instance in a slot in rotating shaft (4) to be spiral-wound on it and extend through an opening (1c1) in fixed part (1c) of the body.

The other end of the ribbon is attached to the outside of fixed part (1c) so as to be suitable for winding on it with the ribbon in the non-deployed position.

The fixed part of body (1c) consists of a circular bearing surface which acts as a stator for ribbon (3). This bearing surface, at either end, has transversal shoulders (1b) for guiding the ribbon gradually as it is extended or wound in, depending on the direction of rotation of rotating drive shaft (4).

In one embodiment, maneuvering device (2) has a rotating knob coupled in a fixed manner, using any known and appropriate means, to rotating drive shaft (4). This rotating knob (2) is therefore mounted at the end of the body, opposite the stator, for winding in measuring ribbon (3). Shaft (4) is mounted free to rotate inside body (1) while being stopped from translating by any known and appropriate means. The length of body assembly (1), especially fixed part (1a) located between maneuvering device (2) and measuring ribbon (3), is determined according to the length of the trocar, with the aim, of measuring ribbon (3) being engaged in the aortic ring.

According to another characteristic, the device has visual indication means of the diameter measured by the ribbon, accordingly corresponding to the deployment of the said ribbon. For example, these visual indication means could be arranged at the ribbon to be read by an endoscope or, in another embodiment, the visual indication means consists of a graduation (2a) on body (1) at maneuvering device (2), in such a way that this graduation can be brought to correspond to a window in maneuvering device (FIG. 3).

The use of the device, according to the characteristics of the invention, is particularly simple, reliable and efficient.

The device is inserted into the aortic conduit through a trocar (not shown). The measuring means of the device, comprising ribbon (3) is placed at the aortic valve to be replaced. The surgeon can then adjust shaft (4) whose rotation causes ribbon (3) to extend. The surgeon turns the maneuvering knob (2) to increase the diameter of ribbon (3) until it comes into contact with the walls of the aortic valve. As soon as rotation is no longer possible, resulting in resistance, the diameter of the valve is determined. The surgeon can then read the graduations indicated previously. Accordingly, measuring ribbon (3) increases gradually from a diameter of 18 mm (FIG. 2), the position in which the ribbon is wound entirely about the shaft (4) and of stator (1c), to a maximum diameter of 40 mm for instance (FIG. 5).

The body of the device can be made of various materials so as to be a single purpose or reusable unit. Similarly, it can be flexible or rigid to allow easier handling. As far as the ribbon is concerned, it is made from a nonelastic flexible film. For instance, it could be made of PTFE.

The advantages are clearly evident from the description, in particular, underlining and reminding about the possibility of being able to use a trocar and accordingly a mini-invasive surgical technique to measure the diameter of the aortic valve to be replaced, an advantageous application in the field of endoscopic surgery.

1. A measuring device for determining the diameter of an aortic valve characterized in that it includes a body of a generally cylindrical shape designed to be engaged into a trocar inserted into an aortic conduit, said body having at one of its ends accessible from the outside of the trocar, a maneuvering device connected to the other end of body, to a measuring device comprising a flexible ribbon mounted in conjunction with a rotating shaft operated by said maneuvering device and a fixed part of the body arranged to be concentric with said shaft and from which said ribbon is deployed under the effect of action on the said maneuvering device to increase the diameter of the ribbon until it comes into contact with the walls of the aortic valve until such a time as rotation becomes impossible, generating resistance corresponding to the diameter of the valve.

2. The device according to claim 1 characterized in that one end of the ribbon is attached in a slot which has a rotating shaft in order to be wound onto it and extend through an opening in the fixed part of the body so as to be attached at the other end to the outside of the said part by being wound on it, corresponding to the non-deployed position of the ribbon.

3. The device according to claim 2 characterized in that the fixed part of the body is constituted by a circular bearing surface which serves as a stator, said bearing surface having transversal shoulders for guiding the ribbon.

4. The device according to claim 1 characterized in that the maneuvering device is a rotating knob mounted to the end of said body opposite the fixed winding part of the ribbon.

5. The device according to claim 4 characterized in that the knob is coupled to the winding shaft mounted free to rotate inside body and locked in translation in the said body.

6. The device according to claim 1 characterized in that it has a visual indicator of the measured diameter corresponding to the deployment of the ribbon.

7. The device according to claim 6 characterized in that the visual indicator is arranged on the ribbon in order to be read by an endoscope.

8. The device according to claim 6 characterized in that the visual indicator comprises a graduation arranged on the body, at the maneuvering device.

9. The device according to claim 8 characterized in that the graduation is brought into alignment with a window that comprises the maneuvering device.
